(19) United States
(12) Patent Application Publication
(10) Pub. No.: US 2014/0350382 A1
(43) Pub. Date: Nov. 27, 2014

(54) APPARATUS AND METHOD FOR ADMINISTERING CONTRAST MEDIUM DURING MAGNETIC RESONANCE TOMOGRAPHY, MAGNETIC RESONANCE TOMOGRAPHY SYSTEM AND USE OF THE APPARATUS

(71) Applicant: SIEMENS AKTIENGESELLSCHAFT, München (DE)

(72) Inventor: Hartmut Richthammer, Regensburg (DE)

(73) Assignee: SIEMENS AKTIENGESELLSCHAFT, München (DE)

(21) Appl. No.: 14/277,096

(22) Filed: May 14, 2014

(30) Foreign Application Priority Data
May 27, 2013 (DE) 102013209832.4

(51) Int. Cl. A61M 5/00; A61B 5/055

(52) U.S. Cl. CPC A61M 5/007; A61B 5/055; A61M 2202/0476
USPC 600/411; 600/420

(57) ABSTRACT

An apparatus and a method are specified for administering a gentle contrast medium during magnetic resonance tomography. The apparatus comprises means for injection of blood into the patient as well as an enrichment device for enriching the blood with the gaseous contrast medium. As part of the magnetic resonance tomography imaging the blood is enriched with the gaseous contrast medium and injected into the patient. The contrast medium contains oxygen.

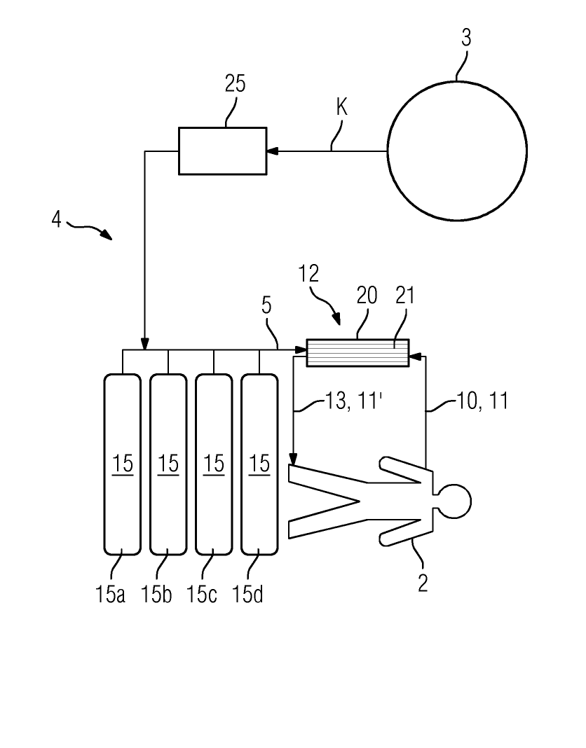

### CROSS-REFERENCE TO RELATED APPLICATIONS

This application claims priority to DE Application No. 102013209832.4, having a filing date of May 27, 2013, the entire contents of which are hereby incorporated by reference.

### FIELD OF TECHNOLOGY

The following relates to an apparatus for administering a contrast medium during magnetic resonance tomography (MRT), as well as to an associated method. The following further relates to an MRT system which comprises such an apparatus.

### BACKGROUND

It is known that magnetic resonance tomography involves an (especially medical) imaging method, in which nuclear spins of the atomic nuclei of the body tissue are excited resonantly by means of a high-frequency magnetic alternating field in a (human or animal) body to be examined. The magnetizing resulting from the excitation induces a magnetic resonance signal (MR signal) in a measurement coil.

### SUMMARY

For image creation, a grayscale value is often computed from the MR signal for each observed volume element, to which a 3D image point (voxel) of a magnetic resonance image (MR image) representing the volume element is assigned. In such cases the corresponding grayscale values of the voxels correspond to the different tissue properties of the respective volume element in each case, which are expressed for example in different relaxation times of the excited nuclear spin.

In order to improve the image quality of an MR image, especially to increase the contrast in the MR image, a contrast medium is typically administered at regular intervals to a patient to be examined during an MRT examination.

Liquid contrast media or contrast media present as a suspension normally used are however sometimes difficult to tolerate for the patients. In particular a contrast medium intolerance in the patient can sometimes induce complications with serious, in the worst case even fatal, consequences.

As part of research into anesthetics, it is proposed in WO 95/27438 A1 that the spatial distribution of a noble gas in a (human) body be analyzed by means of MRT examination. In this process blood is brought into contact with the noble gas outside the body. The blood in contact with the noble gas is subsequently injected intravenously into the body. The noble gas can also be used as a contrast medium.

The underlying aspects described herein relate to specifying an apparatus which makes it possible, during magnetic resonance tomography imaging, to achieve a good image quality with particularly low health risks to the patient being examined. In particular the risk of complications accompanying the administration of normal contrast media may be avoided.

There is provision to inject blood which is enriched with a (under normal conditions) gaseous contrast medium into a patient during an MRT examination. In this case a contrast medium which contains oxygen is employed.

In this context “injection” or “injecting” generally refers to a parenteral application of the blood. In such cases the injection may be given locally, especially exclusively in a section of the patient's body intended for MRT examination, from which a contrast change in the MR image is instigated for precisely this section of the body.

“Contrast medium” in relation to the described embodiments generally refers to a substance which is suitable, through its presence in the body, for modifying an MR signal created during MRT. A contrast medium can in such cases especially be provided by a mixture of substances comprising a number of individual components. An MR signal created during the MRT examination of a specific (body) tissue under contrast medium influence may be either intensified or reduced compared to a basic signal value without contrast media.

The normal conditions cited here comprise a temperature range into which the normal room temperature and the body temperature of the patient fall (e.g. 15-42° C.), as well as a pressure range into which the normal air pressure and the blood pressure of the patient fall (1±0.5 bar).

The idea underlying the described embodiments, using blood enriched with a gaseous contrast medium during an MRT, advantageously makes it possible to significantly expand the portfolio of the substances available as MR contrast media. In particular however the embodiments make it possible to employ oxygen or a gas containing oxygen as a contrast medium. In this case the embodiments utilize the effect known per se that the MR signal created during MRT examination may vary as a function of the oxygen content of the blood. This effect is generally known as the BOLD (“blood oxygenation level dependent”) contrast and is based on the fact that the magnetic properties of the hemoglobin contained in the blood, in its de-oxygenated form, can differ from its form oxygenated with oxygen. Thus by (especially local) injection of oxygen-saturated (possibly oversaturated) blood, the MR contrast can be advantageously significantly modified in a relevant area of the body. The use of oxygen-enriched blood as a contrast medium may be advantageous and especially easy to tolerate for the patient here.

The embodiment for administering a contrast medium during an MRT examination comprises means for injecting blood into the patient to be examined. In addition the embodiment comprises an administration device which may be configured to enrich (to apply or to mix) the blood to be injected with a (under normal conditions) gaseous contrast medium and prepare it for injection into the blood.

The enriched blood may be injected locally in the area of the body to be examined by magnetic resonance tomography for local contrast modification. In this case it may be especially possible to modify the MR image contrast in a subarea of the overall body volume captured by magnetic resonance tomography.

In principle third-party blood can also be used within the framework of the embodiments. However for especially high tolerability for the patient, the patient's own blood may be used. Within the framework of the embodiments this can in principle be provided from stored blood previously taken. The blood of the patient may be however taken directly before or during the course of the MRT examination, enriched with the gaseous contrast medium and injected back into the patient's body. The embodiment accordingly comprises an exemplary embodiment, with means for taking the patient's blood to be enriched and for conveying the blood taken to the enrichment device.

In a form of embodiment especially advantageous in respect of contrast medium administration, the enrichment device may be embodied to enrich the blood with the gaseous contrast medium in a membrane exchange method. Here the enrichment device has at least one semi-permeable (exchange) membrane permeable for the contrast medium, which forms a partition between blood and contrast medium. The gaseous contrast medium diffuses in this case partly pressure-driven through the membrane, while the blood is held back by the membrane.

In an advantageous embodiment the enrichment device may be formed in this case by at least one hollow fiber module. In such a hollow fiber module a plurality of hollow fiber membranes (or capillary membranes) may be accommodated in a per se known manner in a typically tubular module, wherein the hollow fibers are sealed at both end sides against hydraulic short-circuit.

The enrichment device may be embodied as a single-use product which may be discarded after being used once. This avoids the need to sterilize the enrichment device for further use. Preparing the apparatus for further use may be significantly simplified thereby.

In a simple, precise and safe-to-operate form of embodiment the apparatus comprises a control unit for regulating a contrast value of an MR image which may be created by a magnetic resonance tomograph assigned to the apparatus. In such cases a contrast value generally means a value which characterizes the grayscale value distribution of the MR image. The contrast value can in such cases for example be given by the (especially averaged) difference in the grayscale values between two selected image regions. The two image regions in this case are especially automatically selected or selected manually by a user, so that they actually represent different types of tissue (e.g. bones and soft tissue). Furthermore the contrast value can be given by a contrast-to-noise ratio, whereby the contrast-to-noise ratio designates the difference between the signal-to-noise ratios of two relevant tissue types. Furthermore the contrast value can be given by a contrast scope (dynamic range) of the MR image. In such cases a ratio of a maximum signal intensity (grayscale value) to a minimum signal intensity (grayscale value) within the MR image created is referred to as the contrast scope. Finally the contrast value can be formed by a variable derived from one or more of the aforementioned variables. Expediently the control unit may be assigned image evaluation software which may be configured to compute the contrast value (indirectly from the grayscale values or directly from the MR signal).

The control unit may be configured to adapt a measured value of the contrast value supplied to it as an input variable by variation of a throughflow rate of the contrast medium in the enrichment device and/or by variation of a composition of the gas mixture supplied as contrast medium and/or by variation of the gas pressure (i.e. of the pressure of the gas mixture acting as contrast medium 5) to a predetermined required contrast value in a regulated manner.

The control unit may be especially realized in this case in the form of a microcontroller, which may be part of a standalone module (separate from the MRT device). As an alternative however the control unit can be integrated entirely or partly into the controller of the MRT device.

In addition or as an alternative the control unit may be configured, especially in accordance with a user input relating to patient-specific information such as age, body weight, body fat percentage, to set a desired contrast value of the enrichment device in a controlled manner.

The inventive MRT system for performing magnetic resonance tomography comprises a magnetic resonance tomograph, as well as the inventive apparatus for administering a gaseous contrast medium during magnetic resonance tomography in accordance with the above description.

A particular embodiment may be the use of an (especially conventional per se) apparatus for oxygen enrichment of blood, especially a heart-lung machine or a device for extracorporeal membrane oxygenation (ECMO) for producing and administering blood enriched with a gaseous contrast medium for an MRT examination.

### BRIEF DESCRIPTION

An exemplary embodiment is explained in greater detail below with reference to a drawing.

In this drawing the single FIGURE shows, in a schematicized representation, an MRT system 1 for carrying out a magnetic resonance tomography examination (MRT examination) on a patient 2.

### DETAILED DESCRIPTION

The MRT system 1 comprises a magnetic resonance tomograph (abbreviated below to MRT device 3) as well as an apparatus 4 assigned to the MRT device 3 for application of a gaseous contrast medium 5.

The MRT device 3 images a part of the body to be examined (e.g. thorax) of the patient 2 during the course of the MRT examination. Depending on amplitude, frequency shift and/or relaxation time of the captured MR signal, the MRT device 3 computes a three-dimensional image dataset (MR image). The MR image has a corresponding 3D image point (voxel), which is a grayscale value, for each volume element of the part of the body examined.

The apparatus 4 includes means 10 for taking blood 11 from the patient 2 (comprising a cannula and a tube for conveying the blood taken 11), an enrichment device 12 connected downstream in fluid technology terms from the means 10 for enriching the blood 11 with contrast media 5, and also means 13 for injection of the blood 11′ enriched with contrast media 5 into the part of the body of the patient 2 to be examined (wherein the means 13 comprise a cannula and a tube for conveying the blood 11′ to be injected).

Furthermore the apparatus 4 includes a number of gas flasks 15 as a reservoir for different gases. Shown by way of example here is one gas flask 15 in each case for gases 15a (oxygen), 15b (compressed air), 15c (carbon dioxide), and 15d (nitrogen).

Furthermore peripheral components such as sterile filter, heat exchanger, blood and possibly gas pump etc., which, for the sake of clarity are not explicitly shown, are assigned to the enrichment device 12.

A gas mixture produced by mixing two or more of the gases 15a-15d, is supplied to the enrichment device 12 as the gaseous contrast medium 5.

The enrichment device 12 may be essentially formed by a hollow fiber module. The hollow fiber module comprises a plurality of hollow fiber membranes 21 (or capillaries) disposed in parallel in a tube module 20, through which the contrast medium 5 and the withdrawn blood 11 of the patient 2 flow in opposite directions.

With the aid of the apparatus 4 blood 11 may be taken from the patient 2 and supplied to the enrichment device 12. Through diffusion of the contrast medium 5 through the semi-permeable hollow fiber membranes 21 the blood 11 may be enriched with the contrast medium 5, and finally may be injected locally into an area of the body of the patient 2 to be presented in enhanced contrast, through which the MR contrast may be increased locally here.

In the form of embodiment shown here the apparatus 4 comprises a control unit 25 which may be embodied here as a microcontroller with implemented control software. The control unit 25 may be configured to control the enrichment device 12 in order to create a desired contrast medium concentration in the enriched blood 11′ of the patient 2. For this purpose the control unit 20 sets the throughflow/pressure of the individual gases 15a-15d in the enrichment device 12 via controllable valves (not shown here), and is thus especially also able to set the mixture of individual gases acting as contrast medium 5 by varying the mixture ratio (or the composition or the concentration of the individual gas components). In particular the control unit 25 serves to set a desired oxygen concentration in the mixture supplied to the enrichment device 12.

Optionally the individual valves can also be actuated manually.

The control unit 25 also may be connected to the MRT device 3 for signaling purposes. Here the control unit 25 may be configured to derive a contrast value K of the MR image from the MR image of the patient 2 recorded by means of the MRT device 3 and to undertake the control of the enrichment device 12 as a function of this contrast value K.

The difference between the signal intensities of two adjacent (selected by a user) image regions may be included here for example as the contrast value K. The two image regions are therefore selected by the user such that each image region represents one of two specific tissue types (e.g. bone or soft tissue).

In this case the control unit—taking into account health-relevant restrictions—varies throughflow, composition and/or pressure of the contrast medium 5 in the enrichment device 12 in order to set the contrast value K in a regulated manner to a desired required value.

The subject matter of the embodiments may not be restricted to the exemplary embodiment described here. Instead further forms of embodiment can be derived by the person skilled in the art from the description provided here.

1. An apparatus for administering a gaseous contrast medium during a magnetic resonance tomography examination of a patient, the apparatus comprising:
a parenteral application instrument for injection of blood into a part of the body of the patient to be examined, and also
an enrichment device, which is configured to enrich the blood to be injected with the contrast medium and prepare it for injection into the blood, wherein the contrast medium contains oxygen.

2. The apparatus as claimed in claim 1, further comprising
a blood withdrawal instrument for taking blood of the patient to be examined and for supplying the taken blood to the enrichment device.

3. The apparatus as claimed in claim 1,
wherein the enrichment device is configured for enriching the blood in a membrane exchange method with the gaseous contrast means.

4. The apparatus as claimed in claim 3,
wherein the enrichment device is formed by a hollow fiber membrane module.

5. The apparatus as claimed in claim 1,
with a control unit for regulating the MRT image contrast of a magnetic resonance image recorded by a magnetic resonance tomograph, wherein the control unit is configured to adapt a contrast value of the magnetic resonance image supplied as an input variable by variation
of a throughflow rate of the contrast medium in the enrichment device and/or
of a composition of the contrast medium in the enrichment device and/or
of the gas pressure in the enrichment device to a predetermined required contrast value in a regulated manner.

6. A magnetic resonance tomography system for carrying out a magnetic resonance tomography examination, the system comprising:
a magnetic resonance tomograph, and also an apparatus for administering a contrast medium in accordance with claim 1.

7. A method for administering a gaseous contrast medium during a magnetic resonance tomography examination of a patient,
in which blood is enriched with the contrast medium, and
in which the patient is injected with the blood enriched with the contrast medium,
wherein the contrast medium contains oxygen.

8. The method as claimed in claim 7,
in which blood to be enriched with the gaseous contrast medium and to be re-injected is taken from the patient to be examined.

9. The method as claimed in claim 7,
in which the blood enriched with the contrast medium is injected at an area of the body of the patient subject to magnetic resonance tomography for local contrast modification.

10. The method as claimed in claim 7,
in which a throughflow rate, a composition and/or a gas pressure of the contrast medium in the enrichment device is set as a function of a desired contrast value of a created magnetic resonance image.

11. Use of the apparatus as claimed in claim 1 for producing and administering blood enriched with a gaseous contrast medium during a magnetic resonance tomography examination.
